(19) United States
(12) Patent Application Publication
(10) Pub. No.: US 2021/0378249 A1
(43) Pub. Date: Dec. 9, 2021

(54) An Organic Anti-Mold Bakery Additive

(71) Applicants: Venkata Satya Sarveswara Sairam Kuchimanchi, Secunderabad (IN); Vaishnavi Kuchimanchi, Secunderabad (IN)

(72) Inventors: Venkata Satya Sarveswara Sairam Kuchimanchi, Secunderabad (IN); Vaishnavi Kuchimanchi, Secunderabad (IN)

(21) Appl. No.: 17/431,990

(22) PCT Filed: Aug. 9, 2019
(86) PCT No.: PCT/IN2019/050588

§ 371 Date: Aug. 18, 2021

(30) Foreign Application Priority Data
Apr. 25, 2019 (IN) 201941016358

(51) Int. Cl. A21D 2/18; A23L 3/3508; A21D 4/00; C12N 1/20; C12P 7/52; C12P 7/54; C12P 7/56

(52) U.S. Cl. CPC A21D 2/18; A23L 3/3508; A21D 4/00; A23V 2002/00; C12P 7/52; C12P 7/54; C12P 7/56; C12N 1/20

(57) ABSTRACT

Organic Anti-mold Bakery additive” is a natural mold inhibitory food additive which is produced by fermentation of grain flours like wheat or corn converted from the non-edible grade grains followed by downstream processing steps to extract the natural organic acids as preservative components and filter out all protein content. At a minimal dose of 0.3 to 0.5% will enhance the shelf life of bakery products by 25-30 days. The “microbial source” used for the production of this product is a bacterial consortium of three Propionibacterium strains, which were culturally modified by way of strain improvement through medium optimization experiments for product yield enhancement at the ‘in house R&D section’ of Prathista Industries Limited. The active ingredient in “Organic Anti-mold Bakery additive” is a combination of naturally produced organic salts, including propionates, lactates, acetates that have anti-mold properties along with flavor and texture improving efficiency for the finished product.

### FIELD OF THE INVENTION

The present invention relates to an organic mold inhibitor product, produced naturally through microbial fermentation of carbohydrate sources using a bacterial consortium comprising of Propionibacterium spp. that prevents mold growth on food items and plays a major role in shelf life enhancement of bakery products.

### BACKGROUND OF THE INVENTION

Quality loss and spoilage of food products can be caused by a wide range of physical, chemical, enzymatic, and microbial reactions. Food preservatives are used to avoid spoilage during storage, distribution, retailing, and consumption and thus protect quality as well as extend the shelf life of foods and beverages. Examples of most widely used chemical food preservatives that inhibit microbes include benzoates, nitrites, ascorbates, and sorbates (01).

Benzoates are manufactured from toluene, a petroleum by-product and the amount of benzoates to be added to foods must be carefully controlled (not above 0.05-0.1%) as per Food safety regulations. Benzoates also have inhibitory effects on the yeast growth thereby making it a less preferred additive as a preservative in bakery foods preparations (02). Sorbate preservatives are effective inhibitors of most common microorganisms that can attack the foods, without affecting the taste, color, or flavor.

Phenolic polyenes including natamycin and bacteriocins such as nisin have a proven antimycotic property and hence they also find application as antifungal food preservatives. But, their inhibitory effect on yeasts is not desirable for bread baking applications (03) as in case of benzoates. Owing to greater consumer concern for synthetic chemical additives, food preserved with natural preservative has become more popular (04).

Consumption of bakery products including the bread and cake preparations is an integral part of a staple diet for most of the human population across the globe, but accessibility to freshly baked bread may not always be practically feasible for a large populace of consumers. To make bread last longer on our shelves, various natural and artificial preservatives are available in the market that makes the bread loaves last up to 5-7 days. Moreover, the addition of too much preservative can eventually affect the taste of the baked goods.

The applicant's “Organic Anti-mold Bakery additive” is a multifunctional food preservative, effective in preventing the mold growth and helps to extend the shelf life of bakery products including bread, muffins, and cake preparations. It will inhibit or kill the pathogens as well as food spoilage-causing microorganisms. Regulating the calcium levels available in the product can impart firmness and good texture to the bakery preparations (bread and cakes) without influencing the usual organoleptic properties like flavor or color of the bakery product.

It is a preferable antimicrobial and shelf life enhancer as compared to the chemical preservatives such as benzoates, sorbates, and chemical propionates. The applicant's “Organic Anti-Mold Bakery additive” doesn't have any toxic effects on gut health and is a safe natural alternative while retaining the advantages of shelf life enhancement and imparting the desired aroma along with flavors. When compared to other cultured grain flours and cultured whey products “Organic Anti-mold Bakery additive” is a safe antimicrobial and mold inhibitory product because of being free from allergens like glutens/gliadins etc.

The applicant's “Organic Anti-mold Bakery additive” is capable of providing an enhanced shelf life of 25-30 days to bread preparations at a dosage level, ranging from 0.3-0.5% by weight of the dough-mix/batter. At this dosage, it also provides regulated calcium levels to impart firmness and good texture to the bread as well as cake preparations.

### SUMMARY OF THE INVENTION

The present invention relates to an organic mold inhibitor product, produced naturally through microbial fermentation of carbohydrate sources using a bacterial consortium comprising of Propionibacterium spp. that prevents mold growth on food items and plays a major role in shelf life enhancement of bakery products.

In one embodiment of the present invention, the applicant's product “Organic Anti-mold Bakery additive” is a completely organic, natural mold inhibitory food additive which is produced by fermentation of grain flours like wheat or corn converted from the non-edible grade grains. Further downstream processing of the flour will extract natural organic acids as preservative components and filter out all protein content. At a minimal dose of 0.3 to 0.5% of dough mix or batter, it enhances the shelf life of bakery products up to 25-30 days.

Another embodiment of the present invention relates to a process of production of an organic, natural, mold-inhibitory additive produced through microbial fermentation of natural carbohydrate sources followed by downstream processing steps including filtration and drying.

In one embodiment of the present invention, the “microbial source” used for fermentative production of this product is a bacterial consortium comprising of three Propionibacterium strains, namely “Propionibacterium theonii NCIM 2932, Propionibacterium freudenreichii NCIM 2111; and Propionibacterium shermanii NCIM 5137, which were modified by the way of strain improvement through medium optimization experiments for product yield enhancement at the ‘in house R&D section’ of Prathista Industries Limited.

In another embodiment of the present invention, during the production process, the organic acids produced through fermentation are neutralized by dosing of calcium source and thereby providing calcium fortification in the product. Calcium present in the product is an added advantage to the bakery product prepared with “Organic Anti-mold Bakery additive” as it imparts good texture to the bread and retains consistent texture for more time.

In other embodiment of the present invention, the active ingredient in “Organic Anti-mold Bakery additive” is a combination of naturally produced organic acids, including propionates, lactates, and acetates that have anti-mold properties along with flavor and texture improving efficiency for the finished product.

### BRIEF DESCRIPTION OF DRAWINGS

FIG. 1: Growth and production kinetics of bacterial consortium comprising of strains, Propionibacterium theonii, P. freudenreichii; and P. shermanii during batch fermentation.

FIG. 2: Flow chart of the process for Organic Anti-mold Bakery additive production.

FIG. 3: (A) Line diagram representing the number of mold colonies inhibited by Organic Anti-mold Bakery additive at different temperatures (4° C., 28° C., and 40° C.) with respect to control rye bread without the additive. (B) Visualization of rye bread at 28° C. temperature in the presence and absence of Organic Anti-mold Bakery additive. Statistical analysis was performed for triplicate sample readings with Two way ANOVA followed by Tukey's multiple comparison test. **** if P<0.0001 as per Graph Pad Prism 3.0 software analysis.

FIG. 4: (A) Line diagram representing the number of mold colonies on the surface of the bun in the presence and absence of Organic Anti-mold Bakery additive. (B) Images of buns at room temperature for mold inhibition by Organic Anti-mold Bakery additive. Statistical analysis was performed for triplicate sample readings with Two way ANOVA followed by Sidak's multiple comparison test. **** if P<0.0001 as per Graph Pad Prism 3.0 software analysis.

### DETAILED DESCRIPTION OF THE INVENTION

The present invention relates to an organic mold inhibitor product, produced naturally through microbial fermentation of carbohydrate sources using a bacterial consortium comprising of Propionibacterium spp. that prevents mold growth on food items and plays a major role in shelf life enhancement of bakery products.

In one embodiment of the present invention relates to the applicant's product “Organic Anti-mold Bakery additive” is a completely organic, natural mold inhibitory food additive which is produced by fermentation of grain flours like wheat or corn converted from the non-edible grade grains. Further downstream processing of the flour will extract natural organic acids as preservative components and filter out all protein content. At a minimal dose of 0.3 to 0.5% of dough mix or batter, it enhances the shelf life of bakery products up to 25-30 days.

In another embodiment of the present invention, the “microbial source” used for fermentative production of this product is a bacterial consortium comprising of three Propionibacterium strains, namely “Propionibacterium theonii NCIM 2932, Propionibacterium freudenreichii NCIM 2111; and Propionibacterium shermanii NCIM 5137, which were modified by the way of strain improvement through medium optimization experiments for product yield enhancement at the ‘in house R&D section’ of Prathista Industries Limited.

One of the embodiment of the present invention relates to an organic, natural, mold-inhibitory product produced through microbial fermentation of natural carbohydrate sources followed by downstream processing steps including filtration and drying. This product comprises of naturally produced propionic acid as a major component while also having other natural organic acids namely, lactic, acetic, succinic, gluconic acids which impart antimicrobial properties along with flavor and texture improving efficiency for the finished product. During the production process, the organic acids produced through fermentation are neutralized by dosing of calcium source and thereby providing calcium fortification in the product. Calcium present in the product is an added advantage to the bakery product prepared with “Organic Anti-mold Bakery additive” as it imparts good texture to the bread and retains consistent texture for more time.

The present invention is further explained by the following examples. However, the present invention is not limited to these examples in any manner. The following examples are intended to illustrate the working of disclosure and not intended to take restrictively to apply any limitations on the scope of the present invention. Those persons skilled in the art will understand that the equivalent substitutes to the specific substances described herein, or the corresponding improvements are considered to be within the scope of the invention.

### EXPERIMENTAL DETAILS & RESULTS

### Example 1

### (i) Upstream Process Parameters

A microbial consortium comprising of three improved strains of Propionibacterium spp.—Propionibacterium theonii NCIM 2932, P. freudenreichii NCIM 2111 and P. shermanii NCIM 5137—was used for anaerobic fermentation. Strain improvement for acid tolerance was done in-house over a period of 1 year. Fermentation was carried out on a synthetic medium with the following composition (% w/v):

| Components | % w/v |
|---|---|
| Glucose | 8-10% |
| Yeast extract | 1% |
| Potassium dihydrogen phosphate | 0.1% |
| Diammonium hydrogen phosphate | 0.2% |
| Manganese sulphate | 0.0002% |
| Cobalt chloride | 0.001% |
| Magnesium sulphate | 0.001% |
| Sodium chloride | 0.001% |
| Ferrous sulphate | 0.0005% |

Medium (without glucose) was heat sterilized at 121° C. and 15 psi for 25 min. Glucose was autoclaved separately at 115° C. for 15 min and added aseptically to the rest of the medium. All fermentation studies were carried out in 50 L stirred-tank, Stainless Steel (S.S.) bioreactors. Fermentation was done in batch mode.

The process parameters were as follows:

| Parameter | Value |
|---|---|
| pH | 6.5 ± 0.2 |
| Temperature | 30 ± 2° C. |
| Agitation (RPM) | 100 |
| Nitrogen | 0.3 L/min |

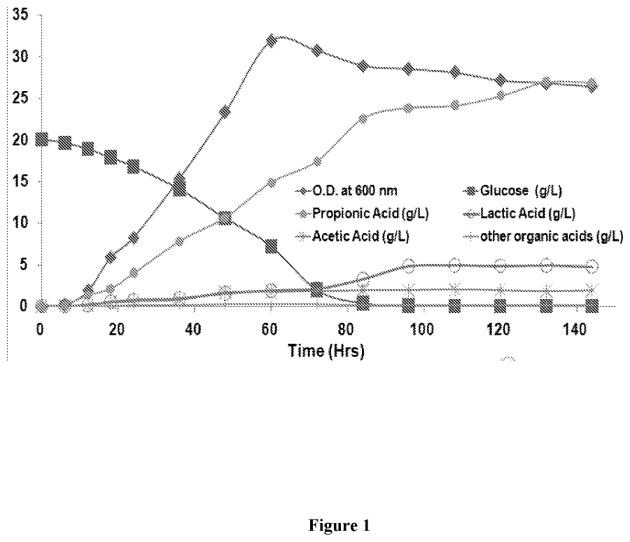

The pH was maintained using 4N HCl and 4N NaOH or ammonium hydroxide in full strength. Temperature and pH were monitored using their respective probes (Sartorius). Sterile nitrogen gas was flushed into the headspace of the reactor using a sterile 0.2 μm PTFE filter (Axiva® 200050 RI, AXIVA Sichem Biotech Pvt. Ltd., India), to maintain anaerobic conditions throughout fermentation. The pre-sterilized fermentation medium in the bioreactor was inoculated with a 10% inoculum of 48 h grown static flask culture. The seed culture was prepared in 500 mL Erlenmeyer flasks, incubated at 30° C. under anaerobic conditions in anaerobic S.S. jars with the help of Whitley Jar Gassing System (Don Whitley Scientific Limited, UK). The progress of one fermentation batch for production of “Organic Anti-mold Bakery additive” is depicted in FIG. 1.

### Example 2

### (ii) In-Process Monitoring of Microbial Growth and Product Yield

Microbial growth during fermentation was measured in terms of optical density (OD) at 600 nm wavelength in a UV-Vis Spectrophotometer (Shimadzu—1800). For dry cell weight estimation, 10-15 mL of fermentation broth was centrifuged at 10,000 rpm for 10 min in a pre-weighed falcon tube. The pellet was dried at 60° C. under vacuum till constant weight was achieved. Organic acids yield were analyzed in the in-process and finished product samples by High-Performance Liquid Chromatography (HPLC).

Analysis of organic acid content involves initial sample preparation, analysis, and calculations. 0.1 g of the test sample was dissolved in 100 mL of HPLC water. Degassing was performed with an ultra-sonicator to prepare the test sample vials. Further, the sample was filtered with a sterile 0.2 μm PTFE filter (Axiva® 200050 RI, AXIVA Sichem Biotech Pvt. Ltd., India). The samples were analyzed with reference to analytic reference standards of respective organic acids.

Further, samples were analyzed by injecting 20 μL of the prepared samples into the HPLC (Shimadzu LC2030 CHT) system. Organic acids column (250×4.6 mm) was used by maintaining column temperature at 30° C. against 8 mM sulfuric acid in water mobile phase. The flow rate was maintained at 0.5 mL/min. while the total run time was 35 min. Detection was performed through UV/Vis at 215 nm.

The standards were injected using the same conditions at concentrations ranging from 2 mM to 20 mM to create a standard curve. Using a spreadsheet application the peak areas of the standards against their concentration were plotted. Further the slope and intercept of the least squares regression line were determined. Checked the line for linearity and discarded the low or high points that are not linear. The test samples were ensured that their absorbance falls within the range of the linear standard concentrations.

Using the Shimadzu Lab Solutions Software, the concentration of respective organic acids in a test sample were determined with reference to the standard calibration curve of respective organic acids in terms of difference of sample peak area and the intercept of gradient of organic acids plotted against the slope of standard curve for each of the individual organic acids.

### Example 3

(iii) Downstream Processing and Product Recovery

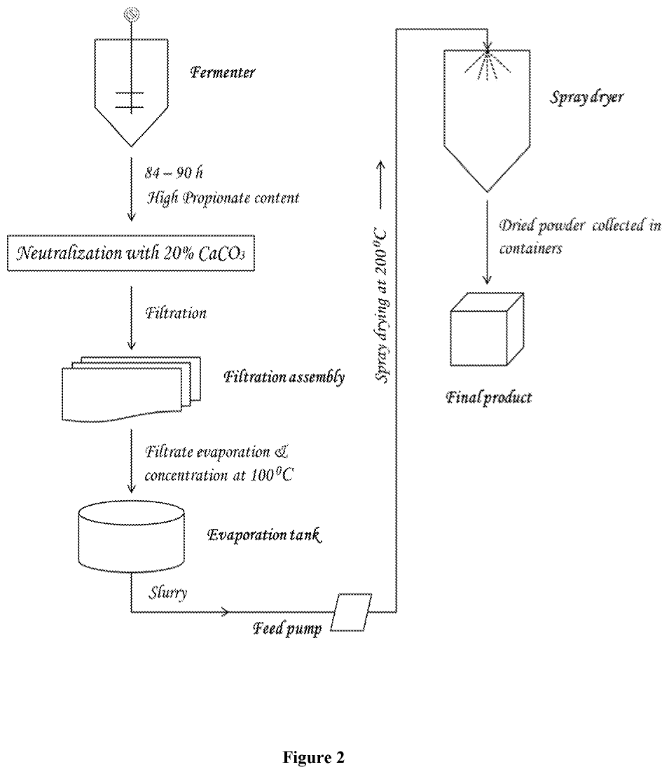

Immediately upon surge of propionic acid production, neutralization step was performed through dosing with pre-sterilized 20% calcium carbonate slurry for the formation of calcium enriched organic salts. As the maximal production of organic acids and complete utilization of Glucose was achieved within 84 h of fermentation, a typical production batch was terminated between 84-90 h of fermentation. Further filtration was performed through 0.3 to 0.4-micron size cloth filters in a plate and frame filtration assembly. The filtered product was concentrated by evaporation and extraction of precipitates followed by homogenization with demineralized water at a high temperature of 95 to 100° C. The slurry was then subjected to spray drying process through the feed inlet at a temperature of 200° C. to obtain “Organic Anti-mold Bakery additive” with desired organic acids levels. The upstream and downstream process has been depicted as a flow chart in FIG. 2.

### Example 4

### (iv) Testing the Product (NMI) for Bakery Products' Shelf-Life Enhancement

During the preparation of dough mix for black (rye) bread and buns, “Organic Anti-mold Bakery additive” was added @ 0.5% w/w of the all-purpose flour content along with other ingredients, before the addition of water and dough kneading. After usual proofing of the dough mix for 60-70 minutes, baking was done for 25 min for bread and 15 min for buns at 200° C. in an Oven Toaster Griller (Morphy Richards). Bread prepared with the additive was considered as ‘Test’. Bread preparation with the same volume of flour and other constituents but without adding any preservative or “Organic Anti-mold Bakery additive” was used as ‘Control’. Both the preparations were prepared and baked under the same conditions for a comparative study.

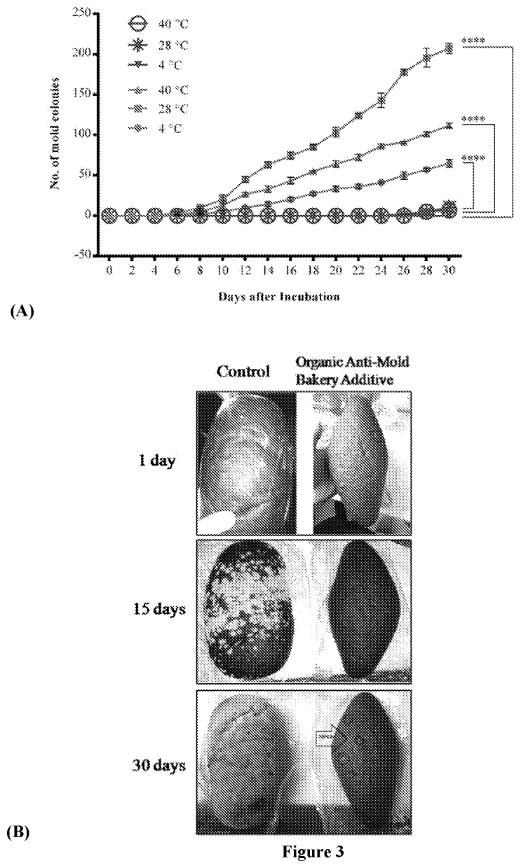

A comparative data of shelf-life of the two bread preparations are presented in Table 1. The ‘Test’, i.e. bread prepared with “Organic Anti-mold Bakery additive”, did not show any mold growth up to 26 days when stored in ambient temperature (25-30° C.) conditions, whereas the ‘Control’ bread exhibited fungal growth by the 5^{th} day. Similarly, the ‘Test’ bread maintained the characteristic texture and flavor throughout the duration of the study. While the ‘Control’ bread turned brittle and off-flavor at the end of 3 days of storage. It was observed, in general, that fungal growth in ‘Control’ was less at the lower and higher ends of the temperature spectrum. Whereas, in ‘Test’, no fungal growth was observed at any temperature (FIG. 3 A-B).

TABLE 1
Comparative analysis of the shelf-life of bread with additive (Test) and
without additive (Control) at different temperatures over a period of
25 days. While no growth was observed in the Test sample,
mold colonies appeared in Control by the 6^{th} day of storage.

| | | No. of Mold Colonies in control | | | No. of Mold Colonies in Test | | |
|---|---|---|---|---|---|---|---|
| S. No | Days | 2-8° C. | 25-30° C. | 40° C. | 2-8° C. | 25-30° C. | 40° C. |
| 1 | 0 | 0 | 0 | 0 | 0 | 0 | 0 |
| 2 | 5 | 0 | 0 | 0 | 0 | 0 | 0 |
| 3 | 10 | 8 | 38 | 18 | 0 | 0 | 0 |
| 4 | 15 | 30 | 102 | 64 | 0 | 0 | 0 |
| 5 | 20 | 68 | 205 | 112 | 0 | 0 | 0 |
| 6 | 25 | 102 | 300 | 170 | 0 | 0 | 0 |

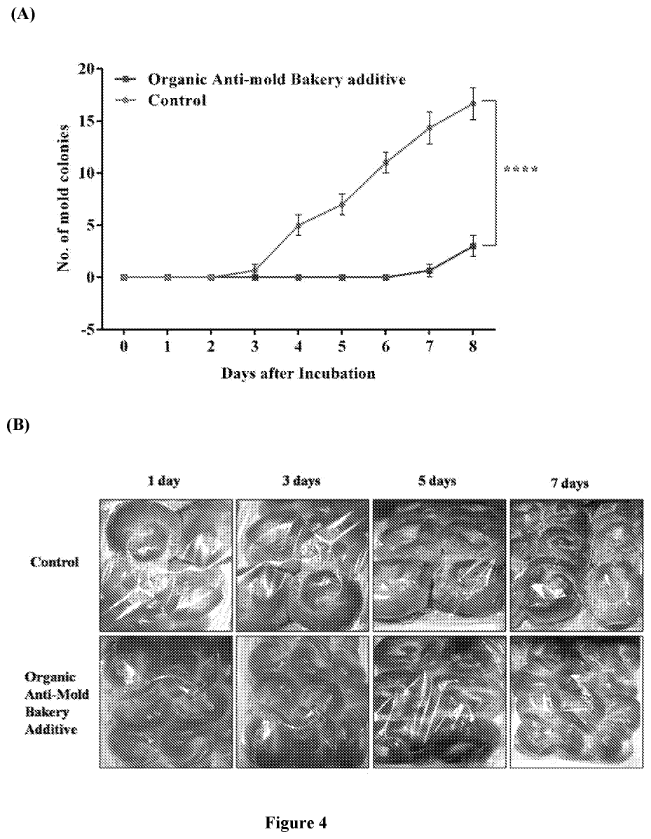

In the case of buns, the “Organic Anti-mold Bakery additive” enhanced the shelf life by 4 more days as compared to ‘Control’ buns where the additive was not added. In ‘Control’ buns, fungal growth was noted from 3 days after preparation while in case of “Organic Anti-mold Bakery additive” added buns initiation of fungal growth was noted at 7 days after preparation (FIG. 4 A-B). The significant increase in the shelf life of buns emended with Organic Anti-mold Bakery additive was noted.

### INDUSTRIAL APPLICABILITY OF THE INVENTION

This invention has applicability in bakery industry as a natural, clean label, ingredient to be used as a preservative for enhancing the shelf life of the bakery products such as bread, muffins, baked donuts, and cakes etc.

### REFERENCES CITED

1) Anand, S. P. and Sati, N. (2013) Artificial preservatives and their harmful effects: looking toward nature for safer alternatives. International Journal of Pharmaceutical Sciences and Research, 4(7), 2496-2501.
2) Inetianbor, J. E., Yakubu, J. M., and Ezeonu, S. C. (2015). Effects of food additives and preservatives on man-a review. Asian Journal of Science and Technology. 6(2), 1118-1135.
3) Deegan, L. H., Cotter, P. D., Hill C., and Ross P. (2006) Bacteriocins: Biological tools for bio-preservation and shelf-life extension. International Dairy Journal, 16(9), 1058-1071.
4) Aneja, K. R., Dhiman, R., Aggarwal, N. K., and Aneja A. (2014) Emerging preservation techniques for controlling spoilage and pathogenic microorganisms in fruit juices. International Journal of Microbiology, Article ID 758942, 1-14.

1. An organic anti-mold bakery additive, a completely organic, natural mold inhibitory food additive which is produced by microbial fermentation of natural carbohydrate sources for enhancing the shelf life of bakery products.

2. The organic anti-mold bakery additive as claimed in claim 1, wherein it comprises of naturally produced propionates as a major component and also having other natural organic salts namely, lactates, acetates, succinates, gluconates which impart antimicrobial properties along with flavor and texture improving efficiency for the finished product.

3. A process of production of an organic, natural, mold-inhibitory additive as claimed in claim 1, which is produced through microbial fermentation of natural carbohydrate sources such as grain flours like wheat or corn converted from the non edible grade grains followed by downstream processing steps including filtration and drying.

4. The process as claimed in claim 3, wherein the microbial source used for fermentative production is a bacterial consortium comprising of three Propionibacterium strains, namely Propionibacterium theonii NCIM 2932, Propionibacterium freudenreichii NCIM 2111; and Propionibacterium shermanii NCIM 5137, which were modified by the way of strain improvement through medium optimization.

5. The process as claimed in claim 3, wherein fermentation is carried out on a synthetic medium comprising of the following composition (% w/v): glucose 8-10%, yeast extract 1%, potassium dihydrogen phosphate 0.1%, diammonium hydrogen phosphate 0.2%, manganese sulphate 0.0002%, cobalt chloride 0.001%, magnesium sulphate 0.001%, sodium chloride 0.001% and ferrous sulphate 0.0005%.

6. The process as claimed in claim 5, wherein medium (without glucose) is heat sterilized at 121° C. and 15 psi for 25 min.; glucose is autoclaved separately at 115° C. for 15 min and added aseptically to rest of the medium.

7. The process as claimed in claim 3, wherein fermentation is carried out in 50 L stirred-tank, Stainless Steel (S.S.) bioreactors in batch mode at temperature 30±2° C., pH 6.5±0.2, agitation 100 rpm and nitrogen 0.3 L/min.

8. The process as claimed in claim 3, wherein neutralization of the organic acids produced through fermentation is done by dosing of calcium source and thereby providing calcium fortification in the product, which imparts good texture to the bread and retains consistent texture for more time.

9. The process as claimed in claim 7, wherein production batch is terminated between 84-90 h of fermentation.

10. The process as claimed in claim 3, wherein filtration is carried out the through 0.3 to 0.4-micron size cloth filters in a plate and frame filtration assembly.

11. The process as claimed in claim 3, wherein the filtered product is concentrated by evaporation and extraction of precipitates followed by homogenization with demineralized water at a high temperature of 95 to 100° C. and spray drying the slurry through the feed inlet at a temperature of 200° C.

12. The organic anti-mold bakery additive as claimed in claim 1, wherein it enhances the shelf-life of bakery products up to 25-30 days at a minimal dose of 0.3 to 0.5% of dough mix or batter.
